(19) United States
(12) Patent Application Publication
(10) Pub. No.: US 2023/0068634 A1
(43) Pub. Date: Mar. 2, 2023

(54) SELECTING ITEMS FOR AN ONLINE CONCIERGE SYSTEM USER TO INCLUDE IN AN ORDER TO ACHIEVE ONE OR MORE NUTRITIONAL GOALS OF THE USER

(71) Applicant: Maplebear Inc. (dba Instacart), San Francisco, CA (US)

(72) Inventors: Chuanwei Ruan, Sunnyvale, CA (US); Peng Qi, Menlo Park, CA (US); Weian Sheng, Foster City, CA (US); Changyao Chen, New York, NY (US); Qiao Jiang, Malden, CA (US)

(21) Appl. No.: 17/462,767

(22) Filed: Aug. 31, 2021

(51) Int. Cl. G06Q 30/06; G16H 20/60; G06Q 50/28; G06F 16/9535; G06N 20/00

(52) U.S. Cl. CPC G06Q 30/0603; G06Q 30/0633; G16H 20/60; G06Q 50/28; G06Q 30/0631; G06Q 30/0623; G06F 16/9535; G06N 20/00

(57) ABSTRACT

An online concierge system allows a user to provide a nutritional goal and uses the nutritional goal as a constraint for selecting candidate orders to display to the user. From prior orders from the user, the online concierge system generates order templates including combinations of generic item descriptions corresponding to items previously included in orders from the user. From the order templates, the online concierge system generates candidate orders including specific items from a warehouse corresponding to the generic item descriptions. The online concierge system selects a set of the candidate orders that each include specific items with combined nutritional information satisfying the user's nutritional goal. Based on probabilities of the user purchasing different candidate orders of the set, the online concierge system selects one or more candidate orders of the set for display to the user.

### BACKGROUND

This disclosure relates generally to ordering one or more items through an online concierge system, and more specifically to selecting one or more items for inclusion in an order accounting for one or more nutritional goals of the user.

In current online concierge systems, shoppers (or “pickers”) fulfill orders at a physical warehouse, such as a retailer, on behalf of users as part of an online shopping concierge service. An online concierge system provides an interface to a user identifying items offered by a physical warehouse and receives selections of one or more items for an order from the user. In current online concierge systems, the shoppers may be sent to various warehouses with instructions to fulfill orders for items, and the shoppers then find the items included in the user order in a warehouse.

Increasingly, users are conscious of nutritional information when purchasing food items. Purchasing food items through an online concierge system affords a user more time to retrieve and to review nutritional information for various food items. For example, a user may more easily retrieve and review nutritional information for different food items form a client device used to access an online concierge system.

While an online concierge system allows a user to more readily review nutritional information for food items, many users lack sufficient nutritional knowledge to select a suitable combination of food items. Additionally, conventional online concierge systems cause users having sufficient nutritional knowledge to expend significant amounts of time to select a combination of food items. For example, a conventional online concierge system causes a user to navigate through multiple interfaces and review multiple search results, while also reviewing nutritional information for various food items, to select food items that satisfy one or more nutritional goals for the user. The time expended by users searching and reviewing food items via an online concierge system to select food items to satisfy one or more nutritional goals for inclusion in an order decreases a likelihood of a user purchasing additional items via the online concierge system and may reduce subsequent user interaction with the online concierge system.

### SUMMARY

An online concierge system maintains information describing nutritional characteristics of various items offered by one or more warehouses. For example, an item catalog received from a warehouse includes an item identifier for each item offered by the warehouse. One or more attributes of an item are associated with the item identifier of the item in the item catalog. Various attributes of an item include nutritional characteristics of the item. Example nutritional characteristics include a number of calories of the item, a number of calories per serving of the item, an amount of protein in the item, an amount of carbohydrates in the item, an amount of fat in the item, amounts of one or more vitamins or minerals included in the item, and any other suitable information describing nutritional content of an item. Hence, the online concierge system maintains information describing nutritional characteristics of items offered by warehouses.

The online concierge system also maintains a set of nutritional goals for users. Each nutritional goal comprises an identifier and a set of nutritional quantities corresponding to the identifier. In various embodiments, an identifier is a textual description describing a goal, and nutritional quantities for achieving the goal. For example, nutritional quantities associated with an identifier specify an amount of a nutritional source per day to achieve a goal corresponding to the identifiers. As an example, an identifier for weight loss is associates with quantities specifying an amount of protein per day, an amount of carbohydrates per day, an amount of fat per day, or amounts of other nutritional sources per day to achieve the weight loss goal. The quantities associated with an identifier may be provided in absolute terms (e.g., a number of grams, a number of calories) or in relative terms (e.g., a percentage of a daily value). The online concierge system may maintain any number of nutritional goals. Additionally, the online concierge system may modify nutritional quantities associated with an identifier over time.

To allow a user to more easily account for nutritional information of items when placing an order for one or more items from a warehouse, the online concierge system receives a nutritional goal from the user and stores the nutritional goal in association with the user. In various embodiments, the online concierge system transmits a questionnaire to the user for display via a customer mobile application. For example, the questionnaire prompts the user to specify a nutritional goal by selecting a nutritional goal of the set maintained by the online concierge system. For example, the questionnaire displays a menu identifying each nutritional goal of the set and receives a selection of a nutritional goal from the user. Alternatively, the questionnaire displays each nutritional goal of the set with a selection element proximate to each nutritional goal and the user selects a selection element proximate to a nutritional goal to identify a selected nutritional goal to the online concierge system. The online concierge system subsequently stores the selected nutritional goal in association with the user to maintain the nutritional goal of the user. Alternatively, the online concierge system receives specific quantities of one or more nutritional sources from the user and stores the nutritional sources and corresponding specific quantities in association with the user as the nutritional goal of the user. In some embodiments, the online concierge system also receives current health statistics for the user (e.g., the user's height, the user's weight) along with the nutritional goal and stores the current health statistic and the received nutritional goal in association with the user. The online concierge system may also receive specific dietary restrictions for the user, such as food allergies of the user or foods the user does not eat. In some embodiments, the restrictions received from the user also specify price constraints, such as a maximum price for an order.

Additionally, the online concierge system generates order templates from orders previously received from the user. In some embodiments, the online concierge system also retrieves one or more recipes and generates the order templates from the retrieved recipes and the orders previously received from the user. When generating order templates, the online concierge system retrieves a taxonomy of items maintained by the online concierge system from item catalogs received from one or more warehouses, with different levels in the taxonomy providing different levels of specificity about items included in the levels. In various embodiments, the taxonomy identifies a generic item description and associates one or more specific items with the generic item identifier. For example, a generic item description identifies “milk,” and the taxonomy associates identifiers of different milk items (e.g., milk offered by different brands, milk having one or more different attributes, etc.), with the generic item identifier. Thus, the taxonomy maintains associations between a generic item description and specific items offered by one or more warehouses matching the generic item description. In some embodiments, different levels in the taxonomy identify items with differing levels of specificity based on any suitable attribute or combination of attributes of the items. For example, different levels of the taxonomy specify different combinations of attributes for items, so items in lower levels of the hierarchical taxonomy have a greater number of attributes, corresponding to greater specificity in a generic item description, while items in higher levels of the hierarchical taxonomy have a fewer number of attributes, corresponding to less specificity in a generic item description. In various embodiments, higher levels in the taxonomy include less detail about items, so greater numbers of items are included in higher levels (e.g., higher levels include a greater number of items satisfying a broader generic item description). Similarly, lower levels in the taxonomy include greater detail about items, so fewer numbers of items are included in the lower levels (e.g., higher levels include a fewer number of items satisfying a more specific generic item description). In some embodiments, the online concierge system maintains different taxonomies for different warehouses, and the online concierge system may receive a taxonomy from a warehouse. In other embodiments, the online concierge system maintains the taxonomy and applies a trained classification model to an item catalog received from the selected warehouse to include different items in levels of the taxonomy, so application of the trained classification model associates specific items with generic item descriptions corresponding to levels within the taxonomy.

To generate an order template, the online concierge system identifies items co-occurring in a threshold number or threshold amount of orders previously received from the user or in recipes obtained by the online concierge system. From the retrieved taxonomy, the online concierge system determines a generic item description corresponding to identified items co-occurring in the threshold number or the threshold amount. Using the retrieved taxonomy allows the online concierge system to determine combinations of generic item descriptions corresponding to combinations of items included in previously received orders from the user or included in recipes obtained by the online concierge system. A combination of generic item descriptions comprises an order template, allowing the order template to specify a combination of generic item descriptions determined from prior orders received from the user or from recipes obtained by the online concierge system. In some embodiments, an order template includes a specific number of generic item descriptions, such as four generic item descriptions, five generic item descriptions, etc.). The online concierge system stores the generated order templates in association with the user, allowing the online concierge system to leverage common occurrences of items in orders previously received from the user or in recipes obtained by the online concierge system to generate order templates for the user that identify combinations of generic item descriptions tailored for the user. In various embodiments, each order template includes a plurality of different generic item descriptions.

When the online concierge system receives a request for an order from the user, the online concierge system retrieves the order templates stored in association with the user and the nutritional goal received from the user. The request for the order includes an identifier of a warehouse for fulfilling the order, so the online concierge system retrieves an item catalog identifying items offered by the identified warehouse. From the items offered by the identified warehouse, a taxonomy (e.g., the previously received taxonomy), the order templates stored in association with the user, and the nutritional goal received from the user, the online concierge system generates candidate orders. To generate a candidate order, the online concierge system identifies specific items offered by the identified warehouse corresponding to each generic item description included in an order template from the taxonomy and the items offered by the identified warehouse. For example, the online concierge system identifies specific items offered by the identified warehouse corresponding to a generic item description and generates different candidate orders that include different specific items offered by the identified warehouse corresponding to a generic item description. Hence, the candidate orders correspond to different combinations of specific items offered by the warehouse that correspond to generic item descriptions from an order template.

The online concierge system selects a set of candidate orders that accounts for the nutritional goal received from the user. From the information describing nutritional characteristics of each specific item included in a candidate order, the online concierge system calculates nutritional information for the candidate order and compares the nutritional information to the candidate order to the nutritional goal received from the user. For example, the online concierge system combines quantities of one or more nutritional sources for each specific item included in a candidate order to calculate nutritional information for the candidate order and compares the combined quantity of the one or more nutritional sources to quantities of the one or more nutritional sources specified by the received nutritional goal. The online concierge system selects candidate orders having nutritional information within a threshold amount of the received nutritional goal for the set in various embodiments. As another example, the online concierge system selects candidate orders having nutritional information that does not exceed the received nutritional goal for the set. This allows the online concierge system to leverage the stored nutritional information for various specific items to select combinations of specific items that satisfy the nutritional goal received from the user. Hence, the online concierge system simplifies accounting for nutritional information of various specific items in comparison to the received nutritional goal to select specific items for inclusion in the order that, in combination, satisfy the nutritional goal received from the user.

In various embodiments, the online concierge also accounts for dietary restrictions received from the user so the set of candidate orders does not include candidate orders including specific items that the user does not eat. Similarly, the online concierge system compares a price for a candidate order (determined from combining prices of specific items included in the candidate order) to a price constraint received from the user and selects candidate orders having prices that do not exceed the price constraint for the set. Further, the online concierge system accounts for availabilities of specific items included in a candidate order determined from application of a machine-learned item availability model to a combination of the specific item and the identified warehouse. In various embodiments, the online concierge system selects candidate orders where each specific item has at least a threshold availability at the identified warehouse for the set.

For each candidate order of the set, the online concierge system determines a likelihood of the user purchasing the candidate order in its entirety. Hence, the likelihood of the user purchasing a candidate order is based on the user purchasing all of the specific items included in the candidate order. In some embodiments, the online concierge system applies a trained purchase model to each specific item of a candidate order. The trained purchase model outputs a probability of the user purchasing an item. The trained purchase model accounts for times when the user previously purchased an item, such as a relative time from a previously received order including the item to a time when the trained purchase model is applied, as well as attributes of the item (e.g., a type of the item, a quantity or an amount of the item that was previously purchased, a brand of the item). The trained purchase model may include a decay constant that decreases a weighting of purchases of the items over time, so purchases of the item at longer time intervals from the time when the trained purchase model is applied have lower weights than weights of purchases at the item at shorter time intervals from the time when the trained purchase model is applied. Additionally, the trained purchase model accounts for a frequency with which the user purchases an item, which increases a likelihood of the user purchasing an item if the receiving user more frequently purchases the item. Other example factors used by the trained purchase model to determine the likelihood of a user purchasing an item include: a time interval between prior orders including the item received from the user, a frequency with which the item is included in prior orders received from the user, times when orders including the item were previously received from the user, preferences of the receiving user, and any other suitable information. The trained purchase model may be trained using any suitable method or combination of methods (e.g., supervised learning, unsupervised learning, semi-supervised learning, etc.).

For a candidate order, the online concierge system applies the trained purchase model to each combination of user and specific item included in the candidate order and determines the likelihood of the user purchasing the candidate order from the probabilities of the user purchasing each specific item included in the candidate order. In some embodiments, the likelihood of the user purchasing the candidate order is an arithmetic mean of the probabilities of the user purchasing each specific item included in the candidate order. As another example, the likelihood of the user purchasing the candidate order is a geometric mean of the probabilities of the user purchasing each specific item included in the candidate order, allowing the online concierge system more significantly penalize a candidate order including a specific item with a low probability of being purchased by the user.

The online concierge system may also account for diversity between specific items included in a candidate order when determining the likelihood of the user purchasing the candidate order. For example, the online concierge system determines similarities between each pair of specific items included in the candidate order and creates a similarity matrix including the determined similarities. In various embodiments, the online concierge system determines a similarity between a specific item and an additional specific item by retrieving embeddings corresponding to the specific item and to the additional specific item and determining a measure of similarity between the embeddings; example measures of similarity include a cosine similarity, a dot product, a Euclidean distance, or any other suitable value. To determine a measure of diversity of the candidate order, the online concierge system calculates a determinant of the similarity matrix. The online concierge system determines the likelihood of the user purchasing the candidate order by combining a value determined from the probabilities of the user purchasing each specific item in the candidate order, as further described above, and the measure of diversity of the candidate order. For example, the likelihood of the user purchasing the candidate order is a sum of the value determined from the probabilities of the user purchasing each specific item in the candidate order, as further described above, and the measure of diversity of the candidate order. In some embodiments, the value determined from the probabilities of the user purchasing each specific item in the candidate order and the measure of diversity of the candidate order may be weighted, and different weights may be applied to the value determined from the probabilities of the user purchasing each specific item in the candidate order, as further described above and to the measure of diversity of the candidate order.

Alternatively, the online concierge system trains a model to receive a candidate order and an identifier of the user as inputs and to output the likelihood of the user purchasing the candidate order. In various embodiments, the online concierge system trains the model from training data that includes a plurality of examples comprising previously received orders from one or more users that are labeled with an indication of whether the user purchased the previously received order or did not purchase the previously received order. The online concierge system 102 stores the set of parameters for trained model and subsequently applies the trained model to a candidate order and the user to determine a likelihood of the user purchasing the complete candidate order.

The online concierge system selects one or more candidate orders of the set based on the likelihoods determined for each candidate order of the set. For example, the online concierge system selects candidate orders of the set having at least a threshold likelihood of being purchased by the user. In other embodiments, the online concierge system ranks the candidate orders of the set based on the determined likelihoods (e.g., so candidate orders of the set having higher likelihoods have higher positions in the ranking) and selects candidate orders having at least a threshold position in the ranking. The online concierge system accounts for total prices of candidate orders of the set when selecting the one or more candidate orders of the set in some embodiments. For example, from information describing specific items in a candidate order of the set, the online concierge system determines a price for each specific item in the candidate order of the set and computes a total price for the candidate order of the set by combining the prices for each specific item in the candidate order of the set. The online concierge system determines an expected value of the candidate order of the set as a product of the likelihood of the user purchasing the candidate order of the set and the total price for the candidate order of the set. Subsequently, the online concierge system selects candidate orders of the set having at least a threshold expected value. Alternatively, the online concierge system ranks candidate orders of the set based on their expected values (e.g., so candidate orders of the set having higher expected values have higher positions in the ranking) and selects candidate orders of the set having at least a threshold position in the ranking.

The online concierge system transmits information identifying the one or more selected candidate orders of the set to a client device of the user. For example, the online concierge system transmits information identifying different selected candidate orders of the set to a client device for display to the user through the customer mobile application. In some embodiments, the information identifying a selected candidate order of the set identifies specific items included in the selected candidate order of the set (e.g., names of the specific items included in the selected candidate order, images of the specific items included in the selected candidate order, descriptions of specific items included in the selected candidate order, or any combination thereof) and a total price of the selected candidate order of the set. A selection element is displayed in conjunction with the information identifying each selected candidate order of the set, in various embodiments. If the online concierge system receives a selection of an interface element displayed in conjunction with information identifying a selected candidate order of the set, the online concierge system generates an order for the user including the specific items included in the selected candidate order of the set, allowing the user to more easily generate an order including multiple specific items that in combination, satisfy the nutritional goal the online concierge system received from the user. This allows the online concierge system to streamline creation of an order by allowing the online concierge system to leverage nutritional information about items offered by the identified warehouse and a nutritional goal received from the user to generate candidate orders that include items with a combination of nutritional information that satisfies the retrieved nutritional goal of the user and to transmit information identifying the generated candidate orders to a user's client device. Hence, the user may select a combination of specific items from an identified candidate order with a single input, or with a reduced number of inputs to identify and to select the items in the identified candidate order.

### BRIEF DESCRIPTION OF THE DRAWINGS

FIG. 1 illustrates an environment of an online shopping concierge service, according to one embodiment.

FIG. 2 is a diagram of an online shopping concierge system, according to one embodiment.

FIG. 3A is a diagram of a customer mobile application (CMA), according to one embodiment.

FIG. 3B is a diagram of a shopper mobile application (SMA), according to one embodiment.

FIG. 4 is flowchart illustrating a process for predicting inventory availability, according to one embodiment.

FIG. 5 is a flowchart of a method for an online concierge system to select one or more items for a user based on one or more nutritional goals of the user, according to one embodiment.

FIG. 6 is a conceptual diagram of a method for an online concierge system selecting one or more items for a user based on one or more nutritional goals of the user, in accordance with an embodiment.

The figures depict embodiments of the present disclosure for purposes of illustration only. One skilled in the art will readily recognize from the following description that alternative embodiments of the structures and methods illustrated herein may be employed without departing from the principles, or benefits touted, of the disclosure described herein.

### DETAILED DESCRIPTION

### System Overview

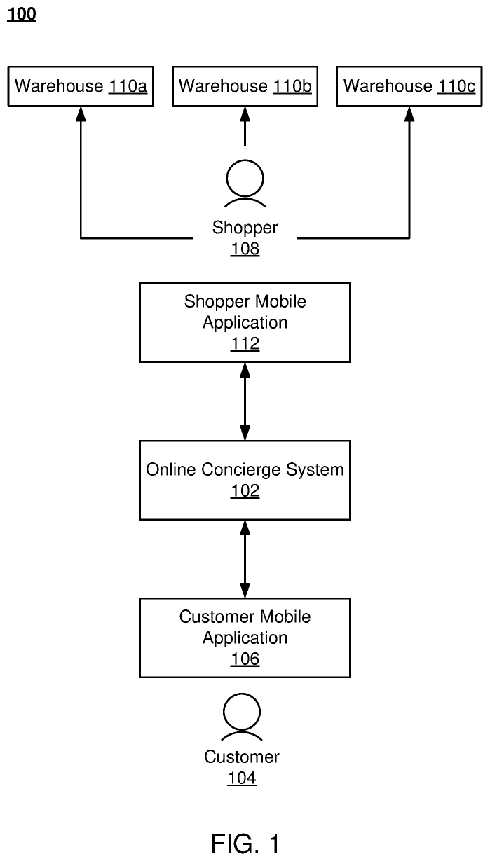

FIG. 1 illustrates an environment 100 of an online platform, according to one embodiment. The figures use like reference numerals to identify like elements. A letter after a reference numeral, such as “110a,” indicates that the text refers specifically to the element having that particular reference numeral. A reference numeral in the text without a following letter, such as “110,” refers to any or all of the elements in the figures bearing that reference numeral. For example, “110” in the text refers to reference numerals “110a” and/or “110b” in the figures.

The environment 100 includes an online concierge system 102. The system 102 is configured to receive orders from one or more users 104 (only one is shown for the sake of simplicity). An order specifies a list of goods (items or products) to be delivered to the user 104. The order also specifies the location to which the goods are to be delivered, and a time window during which the goods should be delivered. In some embodiments, the order specifies one or more retailers from which the selected items should be purchased. The user may use a customer mobile application (CMA) 106 to place the order; the CMA 106 is configured to communicate with the online concierge system 102.

The online concierge system 102 is configured to transmit orders received from users 104 to one or more shoppers 108. A shopper 108 may be a contractor, employee, other person (or entity), robot, or other autonomous device enabled to fulfill orders received by the online concierge system 102. The shopper 108 travels between a warehouse and a delivery location (e.g., the user's home or office). A shopper 108 may travel by car, truck, bicycle, scooter, foot, or other mode of transportation. In some embodiments, the delivery may be partially or fully automated, e.g., using a self-driving car. The environment 100 also includes three warehouses 110a, 110b, and 110c (only three are shown for the sake of simplicity; the environment could include hundreds of warehouses). The warehouses 110 may be physical retailers, such as grocery stores, discount stores, department stores, etc., or non-public warehouses storing items that can be collected and delivered to users. Each shopper 108 fulfills an order received from the online concierge system 102 at one or more warehouses 110, delivers the order to the user 104, or performs both fulfillment and delivery. In one embodiment, shoppers 108 make use of a shopper mobile application 112 which is configured to interact with the online concierge system 102.

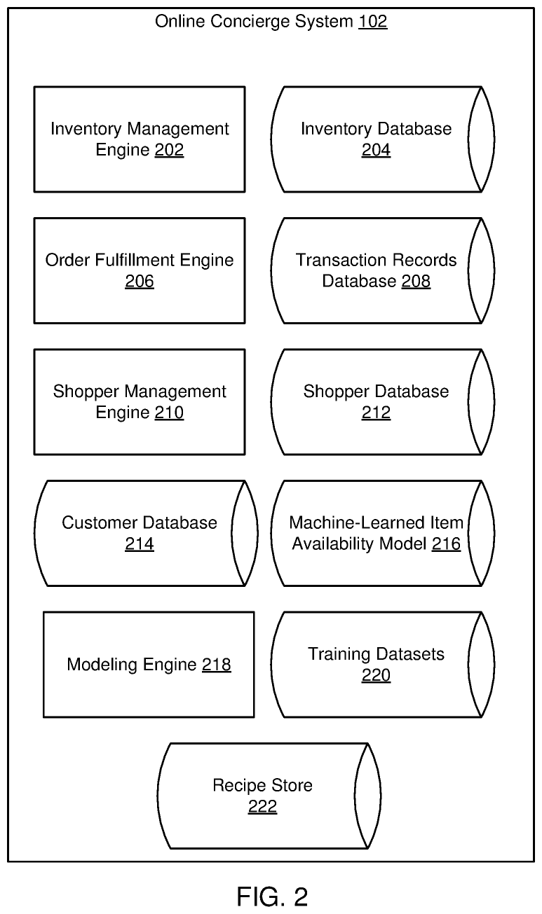

FIG. 2 is a diagram of an online concierge system 102, according to one embodiment. The online concierge system 102 includes an inventory management engine 202, which interacts with inventory systems associated with each warehouse 110. In one embodiment, the inventory management engine 202 requests and receives inventory information maintained by the warehouse 110. The inventory of each warehouse 110 is unique and may change over time. The inventory management engine 202 monitors changes in inventory for each participating warehouse 110. The inventory management engine 202 is also configured to store inventory records in an inventory database 204. The inventory database 204 may store information in separate records—one for each participating warehouse 110—or may consolidate or combine inventory information into a unified record. Inventory information includes both qualitative and qualitative information about items, including size, color, weight, SKU, serial number, and so on. In one embodiment, the inventory database 204 also stores purchasing rules associated with each item, if they exist. For example, age-restricted items such as alcohol and tobacco are flagged accordingly in the inventory database 204. Additional inventory information useful for predicting the availability of items may also be stored in the inventory database 204. For example, for each item-warehouse combination (a particular item at a particular warehouse), the inventory database 204 may store a time that the item was last found, a time that the item was last not found (a shopper looked for the item but could not find it), the rate at which the item is found, and the popularity of the item.

In various embodiments, the inventory management engine 202 maintains a taxonomy of items offered for purchase by one or more warehouses 110. For example, the inventory management engine 202 receives an item catalog from a warehouse 110 identifying items offered for purchase by the warehouse 110. From the item catalog, the inventory management engine 202 determines a taxonomy of items offered by the warehouse 110. different levels in the taxonomy providing different levels of specificity about items included in the levels. In various embodiments, the taxonomy identifies a generic item description and associates one or more specific items with the generic item identifier. For example, a generic item description identifies “milk,” and the taxonomy associates identifiers of different milk items (e.g., milk offered by different brands, milk having one or more different attributes, etc.), with the generic item identifier. Thus, the taxonomy maintains associations between a generic item description and specific items offered by the warehouse 110 marching the generic item description. In some embodiments, different levels in the taxonomy identify items with differing levels of specificity based on any suitable attribute or combination of attributes of the items. For example, different levels of the taxonomy specify different combinations of attributes for items, so items in lower levels of the hierarchical taxonomy have a greater number of attributes, corresponding to greater specificity in a generic item description, while items in higher levels of the hierarchical taxonomy have a fewer number of attributes, corresponding to less specificity in a generic item description. In various embodiments, higher levels in the taxonomy include less detail about items, so greater numbers of items are included in higher levels (e.g., higher levels include a greater number of items satisfying a broader generic item description). Similarly, lower levels in the taxonomy include greater detail about items, so fewer numbers of items are included in the lower levels (e.g., higher levels include a fewer number of items satisfying a more specific generic item description). The taxonomy may be received from a warehouse 110 in various embodiments. In other embodiments, the inventory management engine 202 applies a trained classification module to an item catalog received from a warehouse 110 to include different items in levels of the taxonomy, so application of the trained classification model associates specific items with generic item descriptions corresponding to levels within the taxonomy.

Inventory information provided by the inventory management engine 202 may supplement the training datasets 220. Inventory information provided by the inventory management engine 202 may not necessarily include information about the outcome of picking a delivery order associated with the item, whereas the data within the training datasets 220 is structured to include an outcome of picking a delivery order (e.g., if the item in an order was picked or not picked).

The online concierge system 102 also includes an order fulfillment engine 206 which is configured to synthesize and display an ordering interface to each user 104 (for example, via the customer mobile application 106). The order fulfillment engine 206 is also configured to access the inventory database 204 in order to determine which products are available at which warehouse 110. The order fulfillment engine 206 may supplement the product availability information from the inventory database 204 with an item availability predicted by the machine-learned item availability model 216. The order fulfillment engine 206 determines a sale price for each item ordered by a user 104. Prices set by the order fulfillment engine 206 may or may not be identical to in-store prices determined by retailers (which is the price that users 104 and shoppers 108 would pay at the retail warehouses). The order fulfillment engine 206 also facilitates transactions associated with each order. In one embodiment, the order fulfillment engine 206 charges a payment instrument associated with a user 104 when he/she places an order. The order fulfillment engine 206 may transmit payment information to an external payment gateway or payment processor. The order fulfillment engine 206 stores payment and transactional information associated with each order in a transaction records database 208.

In some embodiments, the order fulfillment engine 206 also shares order details with warehouses 110. For example, after successful fulfillment of an order, the order fulfillment engine 206 may transmit a summary of the order to the appropriate warehouses 110. The summary may indicate the items purchased, the total value of the items, and in some cases, an identity of the shopper 108 and user 104 associated with the transaction. In one embodiment, the order fulfillment engine 206 pushes transaction and/or order details asynchronously to retailer systems. This may be accomplished via use of webhooks, which enable programmatic or system-driven transmission of information between web applications. In another embodiment, retailer systems may be configured to periodically poll the order fulfillment engine 206, which provides detail of all orders which have been processed since the last request.

The order fulfillment engine 206 may interact with a shopper management engine 210, which manages communication with and utilization of shoppers 108. In one embodiment, the shopper management engine 210 receives a new order from the order fulfillment engine 206. The shopper management engine 210 identifies the appropriate warehouse to fulfill the order based on one or more parameters, such as a probability of item availability determined by a machine-learned item availability model 216, the contents of the order, the inventory of the warehouses, and the proximity to the delivery location. The shopper management engine 210 then identifies one or more appropriate shoppers 108 to fulfill the order based on one or more parameters, such as the shoppers' proximity to the appropriate warehouse 110 (and/or to the user 104), his/her familiarity level with that particular warehouse 110, and so on. Additionally, the shopper management engine 210 accesses a shopper database 212 which stores information describing each shopper 108, such as his/her name, gender, rating, previous shopping history, and so on.

As part of fulfilling an order, the order fulfillment engine 206 and/or shopper management engine 210 may access a user database 214 which stores information describing each user. This information could include each user's name, address, gender, shopping preferences, favorite items, stored payment instruments, and so on.

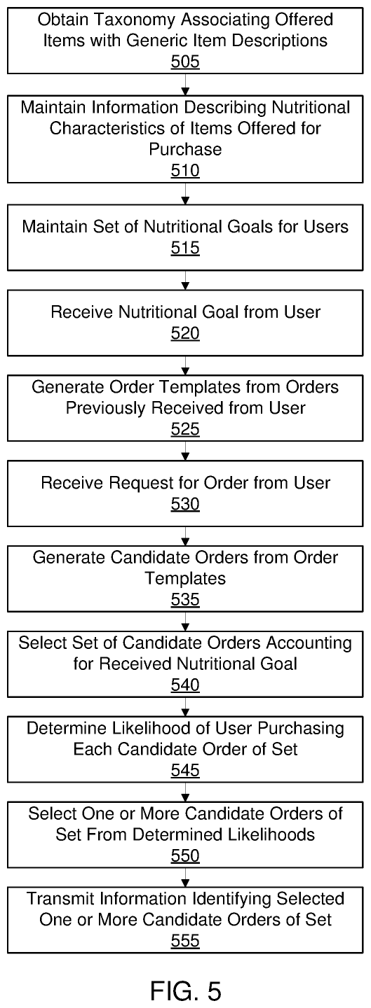

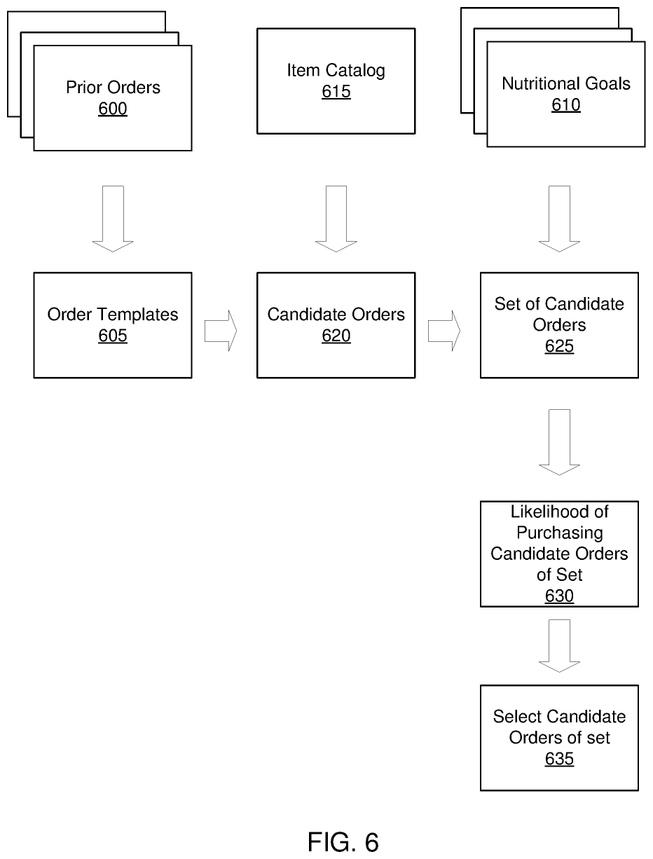

In various embodiments, the order fulfillment engine 206 receives a nutritional goal from a user and leverages the nutritional goal and a taxonomy of items maintained by the inventory management engine 202 to simplify order creation for the user so an order satisfies the received nutritional goal. In various embodiments, the order fulfillment engine 206 receives a nutritional goal from the user, as further described below in conjunction with FIGS. 5 and 6. Additionally, the fulfillment engine 206 retrieves prior orders received from the user from the transaction records database 208 and generates order templates for the user from the taxonomy of items and the prior orders received from the user. In various embodiments, the online concierge system 102 identifies items that co-occur in at least a threshold amount of prior orders from the user and generates order templates that include generic item descriptions from the taxonomy corresponding to the identified items. When generating order templates for the user, the fulfillment engine 206 may account for co-occurrences of items in recipes maintained by a recipe store 222.

When the order fulfillment engine 206 receives a request from a user from whom a nutritional goal was received, the order fulfillment engine 206 retrieves the order templates for the user and an item catalog for a warehouse 110 identified by the request. The order fulfillment engine 206 generates candidate orders for the user, with a candidate order including specific items offered by the warehouse 110 corresponding to different generic item descriptions in an order template. To account for the user's nutritional goal, the order fulfillment engine 206 combines nutritional information for each candidate order, with the nutritional information for a candidate order determined by combining nutritional information for the specific items included in the candidate order. As further described below in conjunction with FIG. 5, the order fulfillment engine 206 selects a set of candidate orders so each candidate order of the set has nutritional information within a threshold amount of the nutritional goal received from the user. To further simplify order creation for the user, the order fulfillment engine 206 selects one or more candidate orders based on likelihood of the user purchasing different candidate orders of the set in their entirety. Hence, for each candidate order of the set, the order fulfillment engine 206 determines a likelihood of the user purchasing the combination of specific items included in a candidate order of the set. Determination of the likelihood of the user purchasing a candidate order in its entirety is further described below in conjunction with FIG. 5. The order fulfillment engine 206 generates an interface for display to the user, such as through the customer mobile application 106 that is populated with information identifying the selected one or more candidate orders of the set. The interface includes an interface element corresponding to various selected candidate orders of the set that, when selected by the receiving user, generates an order including the combination of specific items included in a selected candidate order of the set corresponding to the interface element, simplifying creation of an order by the user that includes a combination of specific items that does not differ from the user's nutritional goal by greater than a threshold amount.

### Machine Learning Models

The online concierge system 102 further includes a machine-learned item availability model 216, a modeling engine 218, training datasets 220, and a recipe store 222. The modeling engine 218 uses the training datasets 220 to generate the machine-learned item availability model 216. The machine-learned item availability model 216 can learn from the training datasets 220, rather than follow only explicitly programmed instructions. The inventory management engine 202, order fulfillment engine 206, and/or shopper management engine 210 can use the machine-learned item availability model 216 to determine a probability that an item is available at a warehouse 110. The machine-learned item availability model 216 may be used to predict item availability for items being displayed to or selected by a user or included in received delivery orders. A single machine-learned item availability model 216 is used to predict the availability of any number of items.

The machine-learned item availability model 216 can be configured to receive as inputs information about an item, the warehouse for picking the item, and the time for picking the item. The machine-learned item availability model 216 may be adapted to receive any information that the modeling engine 218 identifies as indicators of item availability. At minimum, the machine-learned item availability model 216 receives information about an item-warehouse pair, such as an item in a delivery order and a warehouse at which the order could be fulfilled. Items stored in the inventory database 204 may be identified by item identifiers. As described above, various characteristics, some of which are specific to the warehouse (e.g., a time that the item was last found in the warehouse, a time that the item was last not found in the warehouse, the rate at which the item is found, the popularity of the item) may be stored for each item in the inventory database 204. Similarly, each warehouse may be identified by a warehouse identifier and stored in a warehouse database along with information about the warehouse. A particular item at a particular warehouse may be identified using an item identifier and a warehouse identifier. In other embodiments, the item identifier refers to a particular item at a particular warehouse, so that the same item at two different warehouses is associated with two different identifiers. For convenience, both of these options to identify an item at a warehouse are referred to herein as an “item-warehouse pair.” Based on the identifier(s), the online concierge system 102 can extract information about the item and/or warehouse from the inventory database 204 and/or warehouse database and provide this extracted information as inputs to the item availability model 216.

The machine-learned item availability model 216 contains a set of functions generated by the modeling engine 218 from the training datasets 220 that relate the item, warehouse, and timing information, and/or any other relevant inputs, to the probability that the item is available at a warehouse. Thus, for a given item-warehouse pair, the machine-learned item availability model 216 outputs a probability that the item is available at the warehouse. The machine-learned item availability model 216 constructs the relationship between the input item-warehouse pair, timing, and/or any other inputs and the availability probability (also referred to as “availability”) that is generic enough to apply to any number of different item-warehouse pairs. In some embodiments, the probability output by the machine-learned item availability model 216 includes a confidence score. The confidence score may be the error or uncertainty score of the output availability probability and may be calculated using any standard statistical error measurement. In some examples, the confidence score is based in part on whether the item-warehouse pair availability prediction was accurate for previous delivery orders (e.g., if the item was predicted to be available at the warehouse and not found by the shopper, or predicted to be unavailable but found by the shopper). In some examples, the confidence score is based in part on the age of the data for the item, e.g., if availability information has been received within the past hour, or the past day. The set of functions of the item availability model 216 may be updated and adapted following retraining with new training datasets 220. The machine-learned item availability model 216 may be any machine learning model, such as a neural network, boosted tree, gradient boosted tree or random forest model. In some examples, the machine-learned item availability model 216 is generated from XGBoost algorithm.

The item probability generated by the machine-learned item availability model 216 may be used to determine instructions delivered to the user 104 and/or shopper 108, as described in further detail below.

The training datasets 220 relate a variety of different factors to known item availabilities from the outcomes of previous delivery orders (e.g. if an item was previously found or previously unavailable). The training datasets 220 include the items included in previous delivery orders, whether the items in the previous delivery orders were picked, warehouses associated with the previous delivery orders, and a variety of characteristics associated with each of the items (which may be obtained from the inventory database 204). Each piece of data in the training datasets 220 includes the outcome of a previous delivery order (e.g., if the item was picked or not). The item characteristics may be determined by the machine-learned item availability model 216 to be statistically significant factors predictive of the item's availability. For different items, the item characteristics that are predictors of availability may be different. For example, an item type factor might be the best predictor of availability for dairy items, whereas a time of day may be the best predictive factor of availability for vegetables. For each item, the machine-learned item availability model 216 may weight these factors differently, where the weights are a result of a “learning” or training process on the training datasets 220. The training datasets 220 are very large datasets taken across a wide cross section of warehouses, shoppers, items, warehouses, delivery orders, times and item characteristics. The training datasets 220 are large enough to provide a mapping from an item in an order to a probability that the item is available at a warehouse. In addition to previous delivery orders, the training datasets 220 may be supplemented by inventory information provided by the inventory management engine 202. In some examples, the training datasets 220 are historic delivery order information used to train the machine-learned item availability model 216, whereas the inventory information stored in the inventory database 204 include factors input into the machine-learned item availability model 216 to determine an item availability for an item in a newly received delivery order. In some examples, the modeling engine 218 may evaluate the training datasets 220 to compare a single item's availability across multiple warehouses to determine if an item is chronically unavailable. This may indicate that an item is no longer manufactured. The modeling engine 218 may query a warehouse 110 through the inventory management engine 202 for updated item information on these identified items.

Additionally, the modeling engine 218 maintains a trained purchase model that outputs a probability of the user purchasing an item. The trained purchase model accounts for times when the user previously purchased an item, such as a relative time from a previously received order including the item to a time when the model is applied, as well as attributes of the item (e.g., a type of the item, a quantity or an amount of the item that was previously purchased, a brand of the item). The trained purchase model may include a decay constant that decreases a weighting of purchases of the items over time, so purchases of the item at longer time intervals from the time when the trained purchase model is applied have lower weights than weights of purchases at the item at shorter time intervals from the time when the trained purchase model is applied. Additionally, the trained purchase model accounts for a frequency with which the user purchases an item, which increases a likelihood of the user purchasing an item if the user more frequently purchases the item. Other example factors used by the trained purchase model to determine the likelihood of a user purchasing an item include: a time interval between prior orders including the item received from the user, a frequency with which the item is included in prior orders received from the user, times when orders including the item were previously received from the user, preferences of the user, and any other suitable information. The trained purchase model may be trained using any suitable method or combination of methods (e.g., supervised learning, unsupervised learning, semi-supervised learning, etc.).

The recipe store 222 includes information identifying recipes obtained by the online concierge system 102. A recipe includes one or more items, such as a plurality of items, a quantity of each item, and may also include information describing how to combine the items in the recipe. Recipes may be obtained from users, third party systems (e.g., websites, applications), or any other suitable source and stored in the recipe store 222. Additionally, each recipe has one or more attributes describing the recipe. Example attributes of a recipe include an amount of time to prepare the recipe, a complexity of the recipe, nutritional information about the recipe, a genre of the recipe, or any other suitable information. Attributes of a recipe may be included in the recipe by a source from which the recipe was received or may be determined by the online concierge system 102 from items in the recipe or other information included in the recipe.

Additionally, the recipe store 222 maintains a recipe graph identifying connections between recipes in the recipe store 222. A connection between a recipe and another recipe indicates that the connected recipes each have one or more common attributes. In some embodiments, a connection between a recipe and another recipe indicates that a user included items from each connected recipe in a common order or included items from each connected recipe in orders the online concierge system received from the user within a threshold amount of time from each other. In various embodiments, each connection between recipes includes a value, with the value providing an indication of a strength of a connection between the recipes.

### Machine Learning Factors

The training datasets 220 include a time associated with previous delivery orders. In some embodiments, the training datasets 220 include a time of day at which each previous delivery order was placed. Time of day may impact item availability, since during high-volume shopping times, items may become unavailable that are otherwise regularly stocked by warehouses. In addition, availability may be affected by restocking schedules, e.g., if a warehouse mainly restocks at night, item availability at the warehouse will tend to decrease over the course of the day. Additionally, or alternatively, the training datasets 220 include a day of the week previous delivery orders were placed. The day of the week may impact item availability, since popular shopping days may have reduced inventory of items or restocking shipments may be received on particular days. In some embodiments, training datasets 220 include a time interval since an item was previously picked in a previously delivery order. If an item has recently been picked at a warehouse, this may increase the probability that it is still available. If there has been a long time interval since an item has been picked, this may indicate that the probability that it is available for subsequent orders is low or uncertain. In some embodiments, training datasets 220 include a time interval since an item was not found in a previous delivery order. If there has been a short time interval since an item was not found, this may indicate that there is a low probability that the item is available in subsequent delivery orders. And conversely, if there is has been a long time interval since an item was not found, this may indicate that the item may have been restocked and is available for subsequent delivery orders. In some examples, training datasets 220 may also include a rate at which an item is typically found by a shopper at a warehouse, a number of days since inventory information about the item was last received from the inventory management engine 202, a number of times an item was not found in a previous week, or any number of additional rate or time information. The relationships between this time information and item availability are determined by the modeling engine 218 training a machine learning model with the training datasets 220, producing the machine-learned item availability model 216.

The training datasets 220 include item characteristics. In some examples, the item characteristics include a department associated with the item. For example, if the item is yogurt, it is associated with the dairy department. The department may be the bakery, beverage, nonfood and pharmacy, produce and floral, deli, prepared foods, meat, seafood, dairy, the meat department, or dairy department, or any other categorization of items used by the warehouse. The department associated with an item may affect item availability, since different departments have different item turnover rates and inventory levels. In some examples, the item characteristics include an aisle of the warehouse associated with the item. The aisle of the warehouse may affect item availability, since different aisles of a warehouse may be more frequently re-stocked than others. Additionally, or alternatively, the item characteristics include an item popularity score. The item popularity score for an item may be proportional to the number of delivery orders received that include the item. An alternative or additional item popularity score may be provided by a retailer through the inventory management engine 202. In some examples, the item characteristics include a product type associated with the item. For example, if the item is a particular brand of a product, then the product type will be a generic description of the product type, such as “milk” or “eggs.” The product type may affect the item availability, since certain product types may have a higher turnover and re-stocking rate than others or may have larger inventories in the warehouses. In some examples, the item characteristics may include a number of times a shopper was instructed to keep looking for the item after he or she was initially unable to find the item, a total number of delivery orders received for the item, whether or not the product is organic, vegan, gluten free, or any other characteristics associated with an item. The relationships between item characteristics and item availability are determined by the modeling engine 218 training a machine learning model with the training datasets 220, producing the machine-learned item availability model 216.

The training datasets 220 may include additional item characteristics that affect the item availability and can therefore be used to build the machine-learned item availability model 216 relating the delivery order for an item to its predicted availability. The training datasets 220 may be periodically updated with recent previous delivery orders. The training datasets 220 may be updated with item availability information provided directly from shoppers 108. Following updating of the training datasets 220, a modeling engine 218 may retrain a model with the updated training datasets 220 and produce a new machine-learned item availability model 216.

### Customer Mobile Application

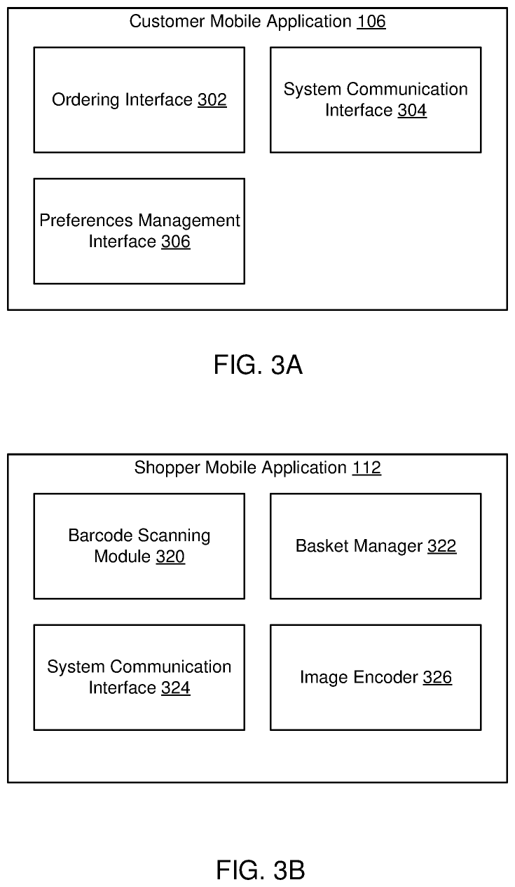

FIG. 3A is a diagram of the customer mobile application (CMA) 106, according to one embodiment. The CMA 106 includes an ordering interface 302, which provides an interactive interface with which the user 104 can browse through and select products and place an order. The CMA 106 also includes a system communication interface 304 which, among other functions, receives inventory information from the online shopping concierge system 102 and transmits order information to the system 102. The CMA 106 also includes a preferences management interface 306 which allows the user 104 to manage basic information associated with his/her account, such as his/her home address and payment instruments. The preferences management interface 306 may also allow the user to manage other details such as his/her favorite or preferred warehouses 110, preferred delivery times, special instructions for delivery, and so on.

### Shopper Mobile Application

FIG. 3B is a diagram of the shopper mobile application (SMA) 112, according to one embodiment. The SMA 112 includes a barcode scanning module 320 which allows a shopper 108 to scan an item at a warehouse 110 (such as a can of soup on the shelf at a grocery store). The barcode scanning module 320 may also include an interface which allows the shopper 108 to manually enter information describing an item (such as its serial number, SKU, quantity and/or weight) if a barcode is not available to be scanned. SMA 112 also includes a basket manager 322 which maintains a running record of items collected by the shopper 108 for purchase at a warehouse 110. This running record of items is commonly known as a “basket”. In one embodiment, the barcode scanning module 320 transmits information describing each item (such as its cost, quantity, weight, etc.) to the basket manager 322, which updates its basket accordingly. The SMA 112 also includes a system communication interface 324 which interacts with the online shopping concierge system 102. For example, the system communication interface 324 receives an order from the system 102 and transmits the contents of a basket of items to the system 102. The SMA 112 also includes an image encoder 326 which encodes the contents of a basket into an image. For example, the image encoder 326 may encode a basket of goods (with an identification of each item) into a QR code which can then be scanned by an employee of the warehouse 110 at check-out.

### Predicting Inventory Availability

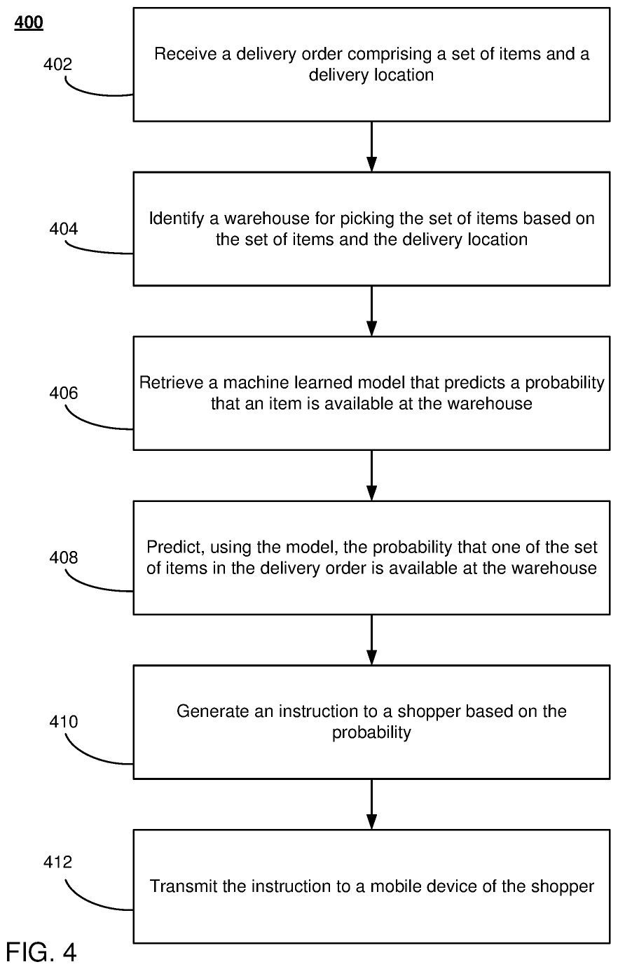

As described with reference to FIG. 2, the machine-learned item availability model 216 of the online concierge system 102 can determine an availability of an item requested by the user 104. FIG. 4 is a flowchart illustrating a process 400 for predicting inventory availability, according to one embodiment. The online concierge system 102 receives 402 a delivery order that includes a set of items and a delivery location. The delivery location may be any location associated with a user, such as a user's home or office. The delivery location may be stored with the user location in the user database 214. Based on the delivery order, the online concierge system 102 identifies a warehouse 404 for picking the set of items in the delivery order based on the set of items and the delivery location. In some cases, the user specifies a particular warehouse or set of warehouses (e.g., a particular grocery store or chain of grocery stores) in the order. In other cases, the online concierge system 102 selects the warehouse based on the items and the delivery location. In some examples, there are a number of different possible warehouses that the set of items may be picked from. The warehouses may be identified by the order fulfillment engine 206 based on warehouses stored by the inventory management engine 202, and warehouses are identified with a suitable inventory and within a threshold distance of the delivery address. In some embodiments, a single delivery order can be split into multiple orders and picked at multiple warehouses, e.g., if the items cannot be fulfilled at a single warehouse. In this example, each possible warehouse is input into the machine-learned item availability model 216.

After the warehouses are identified, the online concierge system 102 retrieves 406 the machine-learned item availability model 216 that predicts a probability that an item is available at the warehouse. The items in the delivery order and the identified warehouses are input into the machine-learned item availability model 216. For example, the online concierge system 102 may input the item, warehouse, and timing characteristics for each item-warehouse pair into the machine-learned item availability model 216 to assess the availability of each item in the delivery order at each potential warehouse at a particular day and/or time. The machine-learned item availability model 216 predicts 408 the probability that one of the set of items in the delivery order is available at the warehouse. If a number of different warehouses are identified 404, then the machine-learned item availability model 216 predicts the item availability for each one. In some examples, the probability that an item is available includes a probability confidence score generated by the machine-learned item availability model 216.

The order fulfillment engine 206 uses the probability to generate 410 an instruction to a shopper. The order fulfillment engine 206 transmits the instruction to the shopper through the SMA 112 via the shopper management engine 210. The instruction is based on the predicted probability. In some examples, the shopper management engine 210 instructs the shopper to pick an item in the delivery order at a warehouse with the highest item availability score. For example, if a warehouse is more likely to have more items in the delivery order available than another warehouse, then the shopper management engine 210 instructs the shopper to pick the item at the warehouse with better availability. In some other examples, the order fulfillment engine 206 sends a message and/or instruction to a user based on the probability predicted by the machine-learned item availability model 216.

### Selecting Items for a User Based on Nutritional Goals of the User

FIG. 5 is a flowchart of one embodiment of a method for an online concierge system 102 to select one or more items for a user based on one or more nutritional goals of the user. In various embodiments, the method includes different or additional steps than those described in conjunction with FIG. 5. Further, in some embodiments, the steps of the method may be performed in different orders than the order described in conjunction with FIG. 5. The method described in conjunction with FIG. 5 may be carried out by the online concierge system 102 in various embodiments.

The online concierge system 102 obtains 505 a taxonomy of items offered by one or more warehouses 110 from item catalogs received from the one or more warehouses 110, with different levels in the taxonomy providing different levels of specificity about items included in the levels. In various embodiments, the taxonomy identifies a generic item description and associates one or more specific items with the generic item identifier. For example, a generic item description identifies “milk,” and the taxonomy associates identifiers of different milk items (e.g., milk offered by different brands, milk having one or more different attributes, etc.), with the generic item identifier. Thus, the taxonomy maintains associations between a generic item description and specific items offered by the warehouse 110 matching the generic item description. In some embodiments, different levels in the taxonomy identify items with differing levels of specificity based on any suitable attribute or combination of attributes of the items. For example, different levels of the taxonomy specify different combinations of attributes for items, so items in lower levels of the hierarchical taxonomy have a greater number of attributes, corresponding to greater specificity in a generic item description, while items in higher levels of the hierarchical taxonomy have a fewer number of attributes, corresponding to less specificity in a generic item description. In various embodiments, higher levels in the taxonomy include less detail about items, so greater numbers of items are included in higher levels (e.g., higher levels include a greater number of items satisfying a broader generic item description). Similarly, lower levels in the taxonomy include greater detail about items, so fewer numbers of items are included in the lower levels (e.g., higher levels include a fewer number of items satisfying a more specific generic item description). The taxonomy may be received from a warehouse 110 in various embodiments. In other embodiments, the online concierge system 102 maintains the taxonomy and applies a trained classification module to an item catalog received from a warehouse 110 to include different items in levels of the taxonomy, so application of the trained classification model associates specific items with generic item descriptions corresponding to levels within the taxonomy.

Additionally, the online concierge system 102 maintains 510 information describing nutritional characteristics of various items offered by one or more warehouses 110. For example, an item catalog received from a warehouse 110 includes an item identifier for each item offered by the warehouse 110. One or more attributes of an item are associated with the item identifier of the item in the item catalog. Various attributes of an item include nutritional characteristics of the item. Example nutritional characteristics include a number of calories of the item, a number of calories per serving of the item, an amount of protein in the item, an amount of carbohydrates in the item, an amount of fat in the item, amounts of one or more vitamins or minerals included in the item, and any other suitable information describing nutritional content of an item. Hence, the online concierge system 102 maintains 510 information describing nutritional characteristics of items offered by warehouses 110.

The online concierge system 102 also maintains 515 a set of nutritional goals for users. Each nutritional goal comprises an identifier and a set of nutritional quantities corresponding to the identifier. In various embodiments, an identifier is a textual description describing a goal, and nutritional quantities for achieving the goal. For example, nutritional quantities associated with an identifier specify an amount of a nutritional source per day to achieve a goal corresponding to the identifiers. As an example, an identifier for weight loss is associates with quantities specifying an amount of protein per day, an amount of carbohydrates per day, an amount of fat per day, or amounts of other nutritional sources per day to achieve the weight loss goal. The quantities associated with an identifier may be provided in absolute terms (e.g., a number of grams, a number of calories) or in relative terms (e.g., a percentage of a daily value). The online concierge system 102 may maintain 515 any number of nutritional goals. Additionally, the online concierge system 102 may modify nutritional quantities associated with an identifier over time.

To allow a user to more easily account for nutritional information of items when placing an order for one or more items from a warehouse 110, the online concierge system 102 receives 520 a nutritional goal from the user and stores the nutritional goal in association with the user. In various embodiments, the online concierge system 110 transmits a questionnaire to the user for display via the customer mobile application 106. For example, the questionnaire prompts the user to specify a nutritional goal by selecting a nutritional goal of the set maintained 515 by the online concierge system 102. For example, the questionnaire displays a menu identifying each nutritional goal of the set and receives a selection of a nutritional goal from the user. Alternatively, the questionnaire displays each nutritional goal of the set with a selection element proximate to each nutritional goal and the user selects a selection element proximate to a nutritional goal to identify a selected nutritional goal to the online concierge system 102. The online concierge system 102 subsequently stores the selected nutritional goal in association with the user to maintain the nutritional goal of the user. Alternatively, the online concierge system 102 receives 520 specific quantities of one or more nutritional sources from the user and stores the nutritional sources and corresponding specific quantities in association with the user as the nutritional goal of the user. In some embodiments, the online concierge system 102 also receives current health statistics for the user (e.g., the user's height, the user's weight) along with the nutritional goal and stores the current health statistic and the received nutritional goal in association with the user. The online concierge system 102 may also receive specific dietary restrictions for the user, such as food allergies of the user or foods the user does not eat. In some embodiments, the restrictions received from the user also specify price constraints, such as a maximum price for an order.

Additionally, the online concierge system 102 generates 525 order templates from orders previously received from the user. In some embodiments, the online concierge system 102 also retrieves one or more recipes and generates 525 the order templates from the retrieved recipes and the orders previously received from the user. When generating 525 order templates, the online concierge system 102 retrieves a taxonomy of items maintained by the online concierge system 102 from item catalogs received from one or more warehouses 110, with different levels in the taxonomy providing different levels of specificity about items included in the levels. In various embodiments, the taxonomy identifies a generic item description and associates one or more specific items with the generic item identifier. For example, a generic item description identifies “milk,” and the taxonomy associates identifiers of different milk items (e.g., milk offered by different brands, milk having one or more different attributes, etc.), with the generic item identifier. Thus, the taxonomy maintains associations between a generic item description and specific items offered by one or more warehouses 110 matching the generic item description. In some embodiments, different levels in the taxonomy identify items with differing levels of specificity based on any suitable attribute or combination of attributes of the items. For example, different levels of the taxonomy specify different combinations of attributes for items, so items in lower levels of the hierarchical taxonomy have a greater number of attributes, corresponding to greater specificity in a generic item description, while items in higher levels of the hierarchical taxonomy have a fewer number of attributes, corresponding to less specificity in a generic item description. In various embodiments, higher levels in the taxonomy include less detail about items, so greater numbers of items are included in higher levels (e.g., higher levels include a greater number of items satisfying a broader generic item description). Similarly, lower levels in the taxonomy include greater detail about items, so fewer numbers of items are included in the lower levels (e.g., higher levels include a fewer number of items satisfying a more specific generic item description). In some embodiments, the online concierge system 102 maintains different taxonomies for different warehouses 110, and the online concierge system 102 may receive a taxonomy from a warehouse 110. In other embodiments, the online concierge system 102 maintains the taxonomy and applies a trained classification model to an item catalog received from the selected warehouse 110 to include different items in levels of the taxonomy, so application of the trained classification model associates specific items with generic item descriptions corresponding to levels within the taxonomy.

To generate 525 an order template, the online concierge system 102 identifies items co-occurring in a threshold number or threshold amount of orders previously received from the user or in recipes obtained by the online concierge system 102. From the retrieved taxonomy, the online concierge system 102 determines a generic item description corresponding to identified items co-occurring in the threshold number or the threshold amount. Using the retrieved taxonomy allows the online concierge system 102 to determine combinations of generic item descriptions corresponding to combinations of items included in previously received orders from the user or included in recipes obtained by the online concierge system 102. A combination of generic item descriptions comprises an order template, allowing the order template to specify a combination of generic item descriptions determined from prior orders received from the user or from recipes obtained by the online concierge system 102. In some embodiments, an order template includes a specific number of generic item descriptions, such as four generic item descriptions, five generic item descriptions, etc.). The online concierge system 102 stores the generated order templates in association with the user, allowing the online concierge system 102 to leverage common occurrences of items in orders previously received from the user or in recipes obtained by the online concierge system 102 to generates 525 order templates for the user that identify combinations of generic item descriptions tailored for the user. In various embodiments, each order template includes a plurality of different generic item descriptions.

When the online concierge system 102 receives 530 a request for an order from the user, the online concierge system 102 retrieves the order templates stored in association with the user and the nutritional goal received from the user. The request for the order includes an identifier of a warehouse 110 for fulfilling the order, so the online concierge system 102 retrieves an item catalog identifying items offered by the identified warehouse 110. From the items offered by the identified warehouse 110, the previously retrieved taxonomy, the order templates stored in association with the user, and the nutritional goal received from the user, the online concierge system 102 generates 535 candidate orders. To generate 535 a candidate order, the online concierge system 102 identifies specific items offered by the identified warehouse corresponding to each generic item description included in an order template from the taxonomy and the items offered by the identified warehouse 110. For example, the online concierge system 102 identifies specific items offered by the identified warehouse 110 corresponding to a generic item description and generates different candidate orders that include different specific items offered by the identified warehouse 110 corresponding to a generic item description. Hence, the candidate orders correspond to different combinations of specific items offered by the warehouse 110 that correspond to generic item descriptions from an order template.

The online concierge system 102 selects 540 a set of candidate orders that accounts for the nutritional goal received from the user. From the information describing nutritional characteristics of each specific item included in a candidate order, the online concierge system 102 calculates nutritional information for the candidate order and compares the nutritional information to the candidate order to the nutritional goal received from the user. For example, the online concierge system 102 combines quantities of one or more nutritional sources for each specific item included in a candidate order to calculate nutritional information for the candidate order and compares the combined quantity of the one or more nutritional sources to quantities of the one or more nutritional sources specified by the received nutritional goal. The online concierge system 102 selects 540 candidate orders having nutritional information within a threshold amount of the received nutritional goal for the set, in various embodiments. As another example, the online concierge system 102 selects 540 candidate orders having nutritional information that does not exceed the received nutritional goal for the set. This allows the online concierge system 102 to leverage the stored nutritional information for various specific items to select combinations of specific items that satisfy the nutritional goal received 520 from the user. Hence, the online concierge system 102 simplifies accounting for nutritional information of various specific items in comparison to the received nutritional goal to select specific items for inclusion in the order that, in combination, satisfy the nutritional goal received 520 from the user.

In various embodiments, the online concierge 102 also accounts for dietary restrictions received from the user so the set of candidate orders does not include candidate orders including specific items that the user does not eat. Similarly, the online concierge system 102 compares a price for a candidate order (determined from combining prices of specific items included in the candidate order) to a price constraint received from the user and selects 540 candidate orders having prices that do not exceed the price constraint for the set. Further, the online concierge system 102 accounts for availabilities of specific items included in a candidate order determined from application of the machine-learned item availability model 216 to a combination of the specific item and the identified warehouse 110. In various embodiments, the online concierge system 102 selects candidate orders where each specific item has at least a threshold availability at the identified warehouse 110 for the set. This allows the set of candidate orders to include candidate orders where each item has at least a threshold availability at the identified warehouse, increasing a likelihood of fulfillment of candidate orders of the set from the identified warehouse 110.

For each candidate order of the set, the online concierge system 102 determines 545 a likelihood of the user purchasing the candidate order in its entirety. Hence, the likelihood of the user purchasing a candidate order is based on the user purchasing all of the specific items included in the candidate order. In some embodiments, the online concierge system 102 applies a trained purchase model to each specific item of a candidate order. The trained purchase model outputs a probability of the user purchasing an item. The trained purchase model accounts for times when the user previously purchased an item, such as a relative time from a previously received order including the item to a time when the trained purchase model is applied, as well as attributes of the item (e.g., a type of the item, a quantity or an amount of the item that was previously purchased, a brand of the item). The trained purchase model may include a decay constant that decreases a weighting of purchases of the items over time, so purchases of the item at longer time intervals from the time when the trained purchase model is applied have lower weights than weights of purchases at the item at shorter time intervals from the time when the trained purchase model is applied. Additionally, the trained purchase model accounts for a frequency with which the user purchases an item, which increases a likelihood of the user purchasing an item if the receiving user more frequently purchases the item. Other example factors used by the trained purchase model to determine the likelihood of a user purchasing an item include: a time interval between prior orders including the item received from the user, a frequency with which the item is included in prior orders received from the user, times when orders including the item were previously received from the user, preferences of the receiving user, and any other suitable information. The trained purchase model may be trained using any suitable method or combination of methods (e.g., supervised learning, unsupervised learning, semi-supervised learning, etc.).

For a candidate order, the online concierge system 102 applies the trained purchase model to each combination of user and specific item included in the candidate order and determines 545 the likelihood of the user purchasing the candidate order from the probabilities of the user purchasing each specific item included in the candidate order. In some embodiments, the likelihood of the user purchasing the candidate order is an arithmetic mean of the probabilities of the user purchasing each specific item included in the candidate order. As another example, the likelihood of the user purchasing the candidate order is a geometric mean of the probabilities of the user purchasing each specific item included in the candidate order, allowing the online concierge system 102 more significantly penalize a candidate order including a specific item with a low probability of being purchased by the user.

The online concierge system 102 may also account for diversity between specific items included in a candidate order when determining 545 the likelihood of the user purchasing the candidate order. For example, the online concierge system 102 determines similarities between each pair of specific items included in the candidate order and creates a similarity matrix including the determined similarities. In various embodiments, the online concierge system 102 determines a similarity between a specific item and an additional specific item by retrieving embeddings corresponding to the specific item and to the additional specific item and determining a measure of similarity between the embeddings; example measures of similarity include a cosine similarity, a dot product, a Euclidean distance, or any other suitable value. To determine a measure of diversity of the candidate order, the online concierge system 102 calculates a determinant of the similarity matrix. The online concierge system determines 545 the likelihood of the user purchasing the candidate order by combining a value determined from the probabilities of the user purchasing each specific item in the candidate order, as further described above, and the measure of diversity of the candidate order. For example, the likelihood of the user purchasing the candidate order is a sum of the value determined from the probabilities of the user purchasing each specific item in the candidate order, as further described above, and the measure of diversity of the candidate order. In some embodiments, the value determined from the probabilities of the user purchasing each specific item in the candidate order and the measure of diversity of the candidate order may be weighted, and different weights may be applied to the value determined from the probabilities of the user purchasing each specific item in the candidate order, as further described above and to the measure of diversity of the candidate order.

Alternatively, the online concierge system 102 trains a model to receive a candidate order and an identifier of the user as inputs and to output the likelihood of the user purchasing the candidate order. In various embodiments, the online concierge system 102 trains the model from training data that includes a plurality of examples comprising previously received orders from one or more users that are labeled with an indication of whether the user purchased the previously received order or did not purchase the previously received order. An example includes embeddings corresponding to each item included in an order and characteristics of the user, such as an embedding for the user.

The online concierge system 102 applies the model, comprising a set of layers of a neural network that are initialized, to each of a plurality of examples of the training data. For an example of the training data, application of the distance prediction model generates a predicted likelihood of a user corresponding to the example purchasing an order corresponding to the example. The online concierge system 102 determines an error term from a difference between the label applied to the example of the training data and the predicted likelihood of the user purchasing the order corresponding to the example. The error term may be generated through any suitable loss function, or combination of loss functions, in various embodiments. For example, the loss function is a mean squared error between a predicted likelihood of the user purchasing an order of an example and a label applied to the corresponding example of the training data. However, in other embodiments, any loss function or combination of loss functions, may be applied to the predicted likelihood of the user purchasing an order of an example and a label applied to the corresponding example of the training data to generate the error term.

The online concierge system 102 repeatedly backpropagates the one or more error terms from the label applied to an example of the training data and the predicted likelihood of the user purchasing an order of the example through layers of a network comprising the distance prediction model. One or more parameters of the network are modified through any suitable technique from the backpropagation of the one or more error terms through the layers of the network. For example, weights between nodes of the network, such as nodes in different layers of the network, are modified to reduce the one or more error terms. The backpropagation of the one or more error terms is repeated by the online concierge system 102 until the one or more loss functions satisfy one or more criteria. For example, the one or more criteria specify conditions for when the backpropagation of the one or more error terms through the layers of the network is stopped. In some embodiments, the online concierge system 102 uses gradient descent or any other suitable process to minimize the one or more error terms in various embodiments.

In response to the one or more loss functions satisfying the one or more criteria and the online concierge system 102 stopping the backpropagation of the one or more error terms, the online concierge system 102 stores the set of parameters for the layers of the neural network. For example, the online concierge system 102 stores the weights of connections between nodes in the network as the set of parameters of the neural network in a non-transitory computer readable storage medium. Hence, training of the model allows the online concierge system 102 to generate and to store a neural network, or other machine learning model, that generates a predicted likelihood of the user purchasing an order. The model may be any machine learning model, such as a neural network, boosted tree, gradient boosted tree, or random forest model in various embodiments. In some examples, the model is trained via a XGBoost process when the model is applied to examples of the training data. The online concierge system 102 retrains the model at various intervals, such as at a periodic interval, in various embodiments, allowing the model to account for changes in purchasing patterns of users.

The online concierge system 102 selects 550 one or more candidate orders of the set based on the likelihoods determined 545 for each candidate order of the set. For example, the online concierge system 102 selects 550 candidate orders of the set having at least a threshold likelihood of being purchased by the user. In other embodiments, the online concierge system 102 ranks the candidate orders of the set based on the determined likelihoods (e.g., so candidate orders of the set having higher likelihoods have higher positions in the ranking) and selects 550 candidate orders having at least a threshold position in the ranking. The online concierge system 102 accounts for total prices of candidate orders of the set when selecting 550 the one or more candidate orders of the set in some embodiments. For example, from information describing specific items in a candidate order of the set, the online concierge system 102 determines a price for each specific item in the candidate order of the set and computes a total price for the candidate order of the set by combining the prices for each specific item in the candidate order of the set. The online concierge system 102 determines an expected value of the candidate order of the set as a product of the likelihood of the user purchasing the candidate order of the set and the total price for the candidate order of the set. Subsequently, the online concierge system 102 selects 550 candidate orders of the set having at least a threshold expected value. Alternatively, the online concierge system 102 ranks candidate orders of the set based on their expected values (e.g., so candidate orders of the set having higher expected values have higher positions in the ranking) and selects 550 candidate orders of the set having at least a threshold position in the ranking.

The online concierge system 102 transmits 555 information identifying the one or more selected candidate orders of the set to a client device of the user. For example, the online concierge system 102 transmits 555 information identifying different selected candidate orders of the set to a client device for display to the user through the customer mobile application 106. In some embodiments, the information identifying a selected candidate order of the set identifies specific items included in the selected candidate order of the set (e.g., names of the specific items included in the selected candidate order, images of the specific items included in the selected candidate order, descriptions of specific items included in the selected candidate order, or any combination thereof) and a total price of the selected candidate order of the set. A selection element is displayed in conjunction with the information identifying each selected candidate order of the set in various embodiments. If the online concierge system 102 receives a selection of an interface element displayed in conjunction with information identifying a selected candidate order of the set, the online concierge system 102 generates an order for the user including the specific items included in the selected candidate order of the set, allowing the user to more easily generate an order including multiple specific items that in combination, satisfy the nutritional goal the online concierge system 102 received 520 from the user. This allows the online concierge system 102 to streamline creation of an order by allowing the online concierge system 102 to leverage nutritional information about items offered by the identified warehouse 110 and a nutritional goal received 520 from the user to generate candidate orders that include items with a combination of nutritional information that satisfies the retrieved nutritional goal of the user and to transmit information identifying the generated candidate orders to a user's client device. Hence, the user may select a combination of specific items from an identified candidate order with a single input, or with a reduced number of inputs to identify and to select the items in the identified candidate order.

FIG. 6 is a process flow diagram of a method for an online concierge system 102 to select one or more items for a user based on one or more nutritional goals of the user. As shown in FIG. 6, the online concierge system 102 maintains prior orders 600 received from a user, with each of the prior orders 600 including one or more items offered for purchase by one or more warehouses 110. As further described above in conjunction with FIG. 5, from items that co-occur in the prior orders 600, the online concierge system 102 generates order templates 605 for the user. To generate an order template 605, the online concierge system 102 identifies items that co-occur in at least a threshold amount of prior orders 600. From a taxonomy maintained by the online concierge system 102, the online concierge system 102 identifies a generic item description corresponding to each identified item and generates order templates 605 that include generic item descriptions corresponding to identified items that co-occurred in at least the threshold amount of prior orders 600. For purposes of illustration, FIG. 6 shows the order templates 605 generated from prior orders 600; however, the online concierge system 102 may account for co-occurrences of items in recipes maintained by the online concierge system 102 to generate the order templates 605, as further described above in conjunction with FIG. 5.

The online concierge system 102 also maintains various nutritional goals 610. As further described above in conjunction with FIG. 5, each nutritional goal comprises an identifier and a set of nutritional quantities corresponding to the identifier. In various embodiments, an identifier is a textual description describing a goal, and nutritional quantities for achieving the goal. For example, nutritional quantities associated with an identifier specify an amount of a nutritional source per day to achieve a goal corresponding to the identifiers. The online concierge system 102 receives a selection of a nutritional goal 610 from the user and stores the selected nutritional goal 610 in association with the user.

After storing the selected nutritional goal 610 in association with the user, the online concierge system 102 receives a request from the user for an order. To allow the user to more easily account for nutritional information of items and select items having nutritional information that satisfies the selected nutritional goal 610, the online concierge system 102 retrieves an item catalog 615 for a warehouse 110 identified by the request from the user. The item catalog 615 includes information describing each item offered for purchase by the warehouse 110 identified by the request, including nutritional information for items offered for purchase by the warehouse 110. Using the order templates 605 generated for the user, the online concierge system 102 generates candidate orders 620 from the item catalog 615. A candidate order 615 includes specific items from the item catalog 615 corresponding to generic item descriptions included in an order template 605. Hence, different candidate orders 615 include different combinations of specific items offered by the warehouse 110 identified by the request that correspond to generic item descriptions included in different order templates 605.

To account for the received nutritional goal 610, the online concierge system 102 selects a set 625 of candidate orders that satisfy the received nutritional goal 610. As further described above in conjunction with FIG. 5, for each candidate order 620, the online concierge system 102 combines nutritional information for the specific items included in a candidate order 620 and compares the combined nutritional information for the specific items to the received nutritional goals 610. Candidate orders 620 having combined nutritional information for their included specific items that satisfy the received nutritional goal 610, or are within a threshold amount of satisfying the nutritional goal 610, are included in the set 625, while candidate orders 620 including specific items with combined nutritional information that is not within a threshold amount of the received nutritional goal 610 are not included in the set 625.

For each candidate order included in the set 625, the online concierge system 102 determines a likelihood 630 of the user purchasing the candidate order included in the set 625. As further described above in conjunction with FIG. 5, the determined likelihood is a likelihood of the user purchasing the candidate order included in the set 625 in its entirety. The online concierge system 102 may account for probabilities of the user purchasing different individual specific items included in a candidate order of the set 625 or may apply a trained model to an embedding or other representation of the candidate order of the set 625 and an embedding or a representation of the user to determine the likelihood 630 of the user purchasing the candidate order of the set 625 in various embodiments. Based on the determined likelihoods 630, the online concierge system 120 selects 635 one or more candidate orders of the set 625 and transmits information identifying the selected one or more candidate orders of the set 625 to a client device of the user for display, as further described above in conjunction with FIG. 5.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium, which include any type of tangible media suitable for storing electronic instructions and coupled to a computer system bus. Furthermore, any computing systems referred to in the specification may include a single processor or may be architectures employing multiple processor designs for increased computing capability.

Embodiments of the invention may also relate to a computer data signal embodied in a carrier wave, where the computer data signal includes any embodiment of a computer program product or other data combination described herein. The computer data signal is a product that is presented in a tangible medium or carrier wave and modulated or otherwise encoded in the carrier wave, which is tangible, and transmitted according to any suitable transmission method.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
obtaining, at an online concierge system, an item catalog from one or more warehouses, an item catalog identifying specific items offered for purchase by a warehouse and including information describing specific items including nutritional information for specific items;
receiving a nutritional goal from a user, the nutritional goal specifying a set of nutritional quantities;
generating, by the online concierge system, order templates for the user from items included in orders the online concierge system previously received from the user;
receiving, at the online concierge system, a request for an order from the user, the request identifying a warehouse for fulfilling the order;
generating, by the online concierge system, candidate orders from the order templates generated for the user and specific items offered by the identified warehouse, each candidate order including a different combination of specific items offered by the identified warehouse;
selecting, by the online concierge system, a set of candidate orders, each candidate order of the set including specific items offered by the identified warehouse that have nutritional information specifying nutritional quantities that, when combined, satisfy the received nutritional goal;
for each candidate order of the set, determining, by the online concierge system, a likelihood of the user purchasing the candidate order of the set in its entirety;
selecting, by the online concierge system, one or more candidate orders of the set based on the determined likelihoods; and
transmitting, by the online concierge system, an interface from the online concierge system to a client device of the user identifying the selected one or more candidate orders of the set.

2. The method of claim 1, wherein generating, by the online concierge system, order templates for the user from items included in orders the online concierge system previously received from the user comprises:
retrieving a taxonomy maintained by the online concierge system, the taxonomy associating one or more generic item descriptions with one or more specific items offered for purchase one or more warehouses;
identifying items co-occurring in at least a threshold amount of the orders the online concierge system previously received from the user;
determining a generic item description corresponding to each of the identified items from the retrieved taxonomy; and
generating an order template including the generic item descriptions corresponding to the identified items.

3. The method of claim 1, wherein generating, by the online concierge system, order templates for the user from items included in orders the online concierge system previously received from the user comprises:
retrieving a taxonomy maintained by the online concierge system, the taxonomy associating one or more generic item descriptions with one or more specific items offered for purchase one or more warehouses;
identifying items co-occurring in at least a threshold amount of the orders the online concierge system previously received from the user or co-occurring in at least a threshold amount of recipes obtained by the online concierge system, each recipe including a plurality of items;
determining a generic item description corresponding to each of the identified items from the retrieved taxonomy; and
generating an order template including the generic item descriptions corresponding to the identified items.

4. The method of claim 1, wherein determining, by the online concierge system, the likelihood of the user purchasing the candidate order of the set in its entirety comprises:
determining a probability of the user purchasing each specific item of the candidate order of the set; and
determining the likelihood of the user purchasing the candidate order of the set in its entirety based on the determined probabilities.

5. The method of claim 4, wherein determining the likelihood of the user purchasing the candidate or der of the set based on the determined probabilities comprises:
determining the likelihood of the user purchasing the candidate order of the set as a geometric mean of the determined probabilities

6. The method of claim 1, wherein determining, by the online concierge system, the likelihood of the user purchasing the candidate order of the set in its entirety comprises:
determining a measure of diversity of the specific items included in the candidate order of the set;
determining a value determined from probabilities of the user purchasing each specific item in the candidate order of the set; and
determining the likelihood of the user purchasing the candidate order of the set in its entirety by combining the measure of diversity and the value.

7. The method of claim 1, wherein determining, by the online concierge system, the likelihood of the user purchasing the candidate order of the set in its entirety comprises:
applying a trained model to the candidate order of the set and to the user, the trained model trained from specific items included in previously received orders received from users and indications of whether the users purchased the previously received orders.

8. The method of claim 1, wherein generating, by the online concierge system, candidate orders from the order templates generated for the user and specific items offered by the identified warehouse comprises:
generating candidate orders from the order templates generated for the user and specific items offered by the identified warehouse, so each candidate order includes a combination of specific items offered by the identified warehouse that satisfies one or more dietary restrictions included in the received nutritional goal and have nutritional information specifying nutritional quantities that, when combined, satisfy the received nutritional goal.

9. The method of claim 1, wherein generating, by the online concierge system, candidate orders from the order templates generated for the user and specific items offered by the identified warehouse comprises:
generating candidate orders from the order templates generated for the user and specific items offered by the identified warehouse, so each candidate order includes a combination of specific items offered by the identified warehouse having a combined price that does not exceed a price constraint included in the received nutritional goal and have nutritional information specifying nutritional quantities that, when combined, satisfy the received nutritional goal.

10. The method of claim 1, wherein selecting, by the online concierge system, one or more candidate orders of the set based on the determined likelihoods comprises:
generating a ranking of the candidate orders of the set based on their determined likelihoods; and
selecting one more candidate orders of the set having at least a threshold position in the ranking.

11. The method of claim 1, wherein selecting, by the online concierge system, one or more candidate orders of the set based on the determined likelihoods comprises:
determining a total price for each candidate order of the set, the total price of a candidate order of the set comprising a combination of prices for specific items included in the candidate order of the set;
determining an expected value for each candidate order of the set, the expected value for the candidate order of the set comprising a product of the total price of the candidate order of the set and a determined likelihood for the candidate order of the set; and
selecting one or more candidate orders of the set based on the expected values.

12. A computer program product comprising a non-transitory computer readable storage medium having instructions encoded thereon that, when executed by a processor, cause the processor to:
obtain, at an online concierge system, an item catalog from one or more warehouses, an item catalog identifying specific items offered for purchase by a warehouse and including information describing specific items including nutritional information for specific items;
receive a nutritional goal from a user, the nutritional goal specifying a set of nutritional quantities;
generate, by the online concierge system, order templates for the user from items included in orders the online concierge system previously received from the user;
receive, at the online concierge system, a request for an order from the user, the request identifying a warehouse for fulfilling the order;
generate, by the online concierge system, candidate orders from the order templates generated for the user and specific items offered by the identified warehouse, each candidate order including a different combination of specific items offered by the identified warehouse;
select, by the online concierge system, a set of candidate orders, each candidate order of the set including specific items offered by the identified warehouse that have nutritional information specifying nutritional quantities that, when combined, satisfy the received nutritional goal;
for each candidate order of the set, determine, by the online concierge system, a likelihood of the user purchasing the candidate order of the set in its entirety;
select, by the online concierge system, one or more candidate orders of the set based on the determined likelihoods; and
transmit, by the online concierge system, an interface from the online concierge system to a client device of the user identifying the selected one or more candidate orders of the set.

13. The computer program product of claim 12, wherein generate, by the online concierge system, order templates for the user from items included in orders the online concierge system previously received from the user comprises:
retrieve a taxonomy maintained by the online concierge system, the taxonomy associating one or more generic item descriptions with one or more specific items offered for purchase one or more warehouses;
identify items co-occurring in at least a threshold amount of the orders the online concierge system previously received from the user;
determine a generic item description corresponding to each of the identified items from the retrieved taxonomy; and
generate an order template including the generic item descriptions corresponding to the identified items.

14. The computer program product of claim 12, wherein generate, by the online concierge system, order templates for the user from items included in orders the online concierge system previously received from the user comprises:
retrieve a taxonomy maintained by the online concierge system, the taxonomy associating one or more generic item descriptions with one or more specific items offered for purchase one or more warehouses;
identify items co-occurring in at least a threshold amount of the orders the online concierge system previously received from the user or co-occurring in at least a threshold amount of recipes obtained by the online concierge system, each recipe including a plurality of items;
determine a generic item description corresponding to each of the identified items from the retrieved taxonomy; and
generate an order template including the generic item descriptions corresponding to the identified items.

15. The computer program product of claim 12, wherein determine, by the online concierge system, the likelihood of the user purchasing the candidate order of the set in its entirety comprises:
determine a probability of the user purchasing each specific item of the candidate order of the set; and
determine the likelihood of the user purchasing the candidate order of the set in its entirety based on the determined probabilities.

16. The computer program product of claim 15, wherein determine the likelihood of the user purchasing the candidate or der of the set based on the determined probabilities comprises:
determine the likelihood of the user purchasing the candidate order of the set as a geometric mean of the determined probabilities

17. The computer program product of claim 12, wherein determine, by the online concierge system, the likelihood of the user purchasing the candidate order of the set in its entirety comprises:
determine a measure of diversity of the specific items included in the candidate order of the set;
determine a value determined from probabilities of the user purchasing each specific item in the candidate order of the set; and
determine the likelihood of the user purchasing the candidate order of the set in its entirety by combining the measure of diversity and the value.

18. The computer program product of claim 12, wherein determine, by the online concierge system, the likelihood of the user purchasing the candidate order of the set in its entirety comprises:
apply a trained model to the candidate order of the set and to the user, the trained model trained from specific items included in previously received orders received from users and indications of whether the users purchased the previously received orders.

19. The computer program product of claim 12, wherein generate, by the online concierge system, candidate orders from the order templates generated for the user and specific items offered by the identified warehouse comprises:
generate candidate orders from the order templates generated for the user and specific items offered by the identified warehouse, so each candidate order includes a combination of specific items offered by the identified warehouse that satisfies one or more dietary restrictions included in the received nutritional goal and have nutritional information specifying nutritional quantities that, when combined, satisfy the received nutritional goal.

20. The computer program product of claim 12, wherein generate, by the online concierge system, candidate orders from the order templates generated for the user and specific items offered by the identified warehouse comprises:
generate candidate orders from the order templates generated for the user and specific items offered by the identified warehouse, so each candidate order includes a combination of specific items offered by the identified warehouse having a combined price that does not exceed a price constraint included in the received nutritional goal and have nutritional information specifying nutritional quantities that, when combined, satisfy the received nutritional goal.

21. The computer program product of claim 12, wherein select, by the online concierge system, one or more candidate orders of the set based on the determined likelihoods comprises:
generate a ranking of the candidate orders of the set based on their determined likelihoods; and
select one more candidate orders of the set having at least a threshold position in the ranking.

22. The computer program product of claim 12, wherein selecting, by the online concierge system, one or more candidate orders of the set based on the determined likelihoods comprises:
determining a total price for each candidate order of the set, the total price of a candidate order of the set comprising a combination of prices for specific items included in the candidate order of the set;
determining an expected value for each candidate order of the set, the expected value for the candidate order of the set comprising a product of the total price of the candidate order of the set and a determined likelihood for the candidate order of the set; and
selecting one or more candidate orders of the set based on the expected values.
